(19) United States
(12) Patent Application Publication
(10) Pub. No.: US 2004/0241839 A1
(43) Pub. Date: Dec. 2, 2004

(54) Culturing neural stem cells

(76) Inventors: Stanislav I. Svetlov, Gainesville, FL (US); Valery G. Kukekov, Gainesville, FL (US)

Correspondence Address: EDWARDS & ANGELL, LLP, P.O. BOX 55874, BOSTON, MA, 02205, US

(21) Appl. No.: 10/821,552

(22) Filed: Apr. 9, 2004

Related U.S. Application Data
(60) Provisional application No. 60/462,357, filed on Apr. 11, 2003.
(60) Provisional application No. 60/463,270, filed on Apr. 16, 2003.

(51) Int. Cl. C12N 5/08; C12N 5/06; A61K 31/739

(52) U.S. Cl. 435/368; 435/354; 514/54

(57) ABSTRACT

The invention provides tissue culture compositions and systems containing lysophosphatidic acid (1-acyl-2-hydroxy-sn-glycero-3-phosphate, LPA) compounds and neural cells such as neural stem cells. Methods for culturing neural stem cells using the compositions promote their differentiation into neurons, oligodendrocytes and astrocytes.

### CROSS-REFERENCE TO RELATED APPLICATIONS

The present application claims the priority of U.S. provisional patent application No. 60/462,357, filed on Apr. 11, 2003; and U.S. provisional application No. 60/463,270, filed on Apr. 16, 2003. The foregoing are incorporated herein by reference in their entirety.

### STATEMENT AS TO FEDERALLY SPONSORED RESEARCH

This invention was made with United States government support under grant number R21 DK61649 awarded by the National Institutes of Health. The United States government may have certain rights in the invention.

### FIELD OF THE INVENTION

The invention relates to the fields of cell-biology and medicine. More particularly, the invention relates to compositions and methods for culturing neural stem cells.

### BACKGROUND

Proliferation and differentiation of neural stem/progenitor cells in normal and injured brain are regulated by a number of growth factors and cytokines, many of which have yet to be identified. The roles for peptide growth factors such as epidermal growth factor (EGF) acting through protein tyrosine kinase receptor (EGFR), or basic fibroblast growth factor (bFGF or FGF2) have been studied extensively (Gritti et al., J. Neurosci. 19:3287-3297, 1999; Vaccarino et al., Neuropsychopharmacology 25:805-815, 2001; and Tiedemann et al., Dev. Growth Differ. 43:469-502, 2001). Moreover, EGF and FGF2 have been incorporated into commercial medium formulations to grow and maintain neural progenitors. In contrast, a role for lysophospholipid growth factors in neural stem/progenitor cell growth and differentiation is largely unexplored.

### SUMMARY

The invention is based on the discovery that lysophosphatidic acid (LPA) is useful for initiating and maintaining neural stem/progenitor cell growth and differentiation in vitro. It is demonstrated herein that neural stem cells can be successfully cultured in medium containing LPA in lieu of EGF and FGF2. The invention thus relates to compositions and methods for culturing neural stem cells in vitro using LPA compounds. Accordingly, in one aspect the invention includes a tissue culture system including: (a) at least one isolated neural cell expressing at least one LPA receptor; (b) a lysophosphatidic acid (LPA) compound; and (c) a basal culture medium. For optimal biological activity, the LPA compound used in the tissue culture system is preferably in the form of LPA 2:5, 18:0 (oleoyl), 16:1 (palmitoyl), or 14:0 (myristoyl), and more preferably 18:1 or 16:0.

In preferred embodiments of the tissue culture system, the isolated neural cell is a stem/progenitor cell. The neural stem/progenitor cell can be situated within a neurosphere. The neural stem/progenitor cell can be derived from a mammal, for example from a mouse or a human.

The LPA receptor expressed by the neural cell used in the tissue culture system can be an LPA1, LPA2, or LPA3 receptor.

The stem/progenitor cell of the tissue culture system can express selected markers, such as at least one of a Sca-1 and an AC133 antigen, and LPA receptors, such as at least one of an LPA1, LPA2 and LPA3 receptor.

The stem/progenitor can cell further expresses at least one marker of neuronal differentiation such as βIII tubulin or nestin.

In another aspect, the invention provides a method of culturing at least one neurosphere from isolated brain cells. The method includes the steps of: (a) providing at least one isolated brain cell; and (b) culturing the brain cell in a medium containing a lysophosphatidic acid (LPA) compound under conditions that allow for growth and differentiation of a neurosphere from the isolated brain cell.

The step (b) of culturing at least one brain cell under conditions that allow for growth of a neurosphere can further allow for proliferation and differentiation of the cells within the neurosphere into at least one cell type including a neuron, an astrocyte, and an oligodendrocyte. In embodiments in which one cell type is a neuron, at least one lineage-specific marker can be expressed by the cell, including βIII tubulin, and nestin and CNPAse.

Yet a further aspect of the invention is an isolated neural cell cultivated in a basal culture medium including a lysophosphatidic acid (LSA) compound. The isolated neural cell can be situated within a neurosphere. Preferred forms of the SPA compound included in the culture medium can have the form of LPA 20:5, 18:1 (oleoyl), 16:0 (palmitoyl), or 14:0 (myristoyl), and more preferably 18:1 or 16:0.

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991; and Lewin, Genes V, Oxford University Press: New York, 1994.

Although methods and materials similar or equivalent to those described herein can be used in the practice or testing of the present invention, suitable methods and materials are described below. All publications, patent applications, patents, and other references mentioned herein are incorporated by reference for the proposition cited. In the case of conflict, the present specification, including definitions will control. The particular embodiments discussed below are illustrative only and not intended to be limiting.

### BRIEF DESCRIPTION OF THE DRAWINGS

FIG. 1 is two micrographs (A, B) showing formation of neurospheres in serum-free semi-solid medium, and two graphs (C, D) showing the effect of LPA and DGPP on the neurosphere size (C) and number (D), according to an embodiment of the invention. (A) shows low power microscopy of typical neurospheres formed 96 hr following plating in the presence of LPA, by Hoffmann modulation contrast, ×10. Inset in (A) shows a phase contrast image of a large neurosphere formed at 96 hr, ×40. (B) shows typical neurospheres produced during 2 weeks of maintenance in cultures containing LPA. In (C, D), neurospheres were maintained in culture for 2 weeks in the presence of LPA alone or LPA plus 1 μM or 50 μM DGPP. The number/field and the size of at least 200 neurospheres formed at 2 weeks were counted. The results are presented as mean +SEM of three independent experiments from four different neurosphere preparations. Unpaired Student's t-test was used to assess significance of the mean difference, and F test was used to compare variances.

FIG. 2(A-I) is a series of nine fluorescence micrographs showing immunocytochemical staining of AC133, and Sca-1 antigens (A-C) and LPA receptors (D-I) in neurospheres produced and grown in the presence of LPA, according to an embodiment of the invention. The cover slips were fixed and examined for the expression of AC133, Sca-1 and LPA receptors using double immunostaining with antibodies against AC133 and Sca-1 antigens, and against LPA1, LPA2 and LPA3 receptors.

FIG. 3 is four fluorescence micrographs showing co-localization of AC133 and Sca-1 with LPA receptors in mouse neurospheres, according to an embodiment of the invention. Fixed neurospheres were double immunostained with AC133 or Sca-1 and LPA1 or LPA3 receptor antibody, followed by development of bound proteins with secondary antibody coupled-with Texas Red or Alexa green. Arrowheads depict the areas of most extensive co-localization. The results shown are representative of three independent experiments from two different neurosphere-preparations.

FIG. 4 is four fluorescence micrographs showing expression and co-localization of LPA receptors and markers of neuronal lineage; i.e., βIII-tubulin and nestin in attached neurospheres grown in the presence of LPA, according to an embodiment of the invention. The neurospheres, produced and maintained for 2 weeks in the presence of LPA, were attached to cover slips as described above and then cultured for an additional week in serum-free medium supplemented with LPA. The cover slips were fixed and double immunostained with antibody against LPA1 or LPA3 receptors and βIII-tubulin or nestin. (A) shows LPA1/βIII-tubulin co-localization; (B) shows LPA1/nestin co-localization; (C) shows LPA3/βIII-tubulin co-localization; (D) shows LPA3/nestin co-localization.

FIG. 5 is four fluorescence micrographs showing expression and co-localization of LPA receptors and markers of oligodendrocytes and astrocyte lineages, i.e., CNPase and GFAP. Neurospheres were grown as above in the presence of LPA, fixed and probed with antibody against LPA1 or LPA3 and CNPase or GFAP using dual immunostaining techniques. (A) shows LPA1/CNPase colocalization; (B) shows LPA1/GFAP co-localization; (C) shows LPA3/CNPase co-localization; and (D) shows LPA3/GFAP lack of colocalization. Arrowheads indicate the most extensive co-localization.

### DETAILED DESCRIPTION

The invention relates to compositions and methods for culturing neural stem cells in vitro. Neural stem cells cultured in LPA-containing medium followed by addition of serum can proliferate and differentiate into oligodendrocytes, neurons and astrocytes. In the experiments described herein using cultured mouse brain neurospheres as a model of developing brain, serum-free basal culture medium containing LPA is shown to effectively support growth of neurospheres in vitro—even in the absence of growth factors such EGF and FGF2, traditionally employed in such media formulations. Neurosphere growth induced by LPA was slower than that induced by EGF and FGF2, was sustained longer (i.e., for up to 3 months), and was associated with proliferation of cells expressing both Sca-1 and AC133 antigens, which are markers of primitive stem cells of hematopoietic and neural origin. Sca-1 and AC133 positive cells within neurospheres were shown to express three known subtypes of the LPA receptor, i.e., LPA1, LPA2 and LPA3. LPA-induced formation and growth of neurospheres was specifically inhibited by diacylglycerol-pyrophosphate (DGPP), an antagonist of LPA1/LPA3 receptors, confirming that the effect of LPA was specifically mediated via these receptors.

Immunocytochemical studies determined that LPA receptors were expressed in developing neurospheres attached to coverslips and grown in the presence of LPA, and that these receptors were co-expressed and/or co-localized with markers of neuronal differentiation, i.e., βIII-tubulin, and nestin, but not with GFAP, a marker of astrocyte lineage. Sca-1 antigen and AC133 were still detected in the residual core of neurospheres grown attached in the presence of LPA, but not EGF/FGF, and co-localized with LPA receptors. Collectively, the data reveal novel properties of LPA that favor initiation and regulation of neural stem cell growth and differentiation in vitro.

Accordingly, the invention provides methods and compositions based on LPA that are useful for initiating, propagating and differentiating cultures of neural stem cells. In one aspect, the invention provides a tissue culture system including: (a) at least one isolated neural cell expressing at least one LPA receptor; (b) a lysophosphatidic acid (LPA) compound; and (c) a basal culture medium.

The below described preferred embodiments illustrate adaptations of these compositions and methods. Nonetheless, from the description of these embodiments, other aspects of the invention can be made and/or practiced based on the description provided below.

### Tissue Culture System

Cell culture techniques are generally known in the art and are described in detail in methodology treatises such as Culture of Animal Cells: A Manual of Basic Technique, 4th edition, by R. Ian Freshney, Wiley-Liss, Hoboken, N.J., 2000; and General Techniques of Cell Culture, by Maureen A. Harrison and Ian F. Rae, Cambridge University Press, Cambridge, UK, 1994.

The invention provides a tissue culture system including at least one neural cell expressing an LPA receptor, an LPA compound, and a basal culture medium. A neural cell expressing an LPA receptor, or capable of induction of such a receptor can be obtained, for example, from the brain of any mammal, including those of embryonic and adult mice and humans.

### LPA Compounds as Bioactive Signaling Molecules

As disclosed in the examples below, the ability of LPA compounds to promote growth and differentiation of neural stem cells is mediated by signaling initiated by LPA receptors on the surface of primitive neural cell stem/progenitor cells undergoing in vitro clonal expansion and differentiation in isolated structures known as neurospheres. In general, the field of phospholipid signaling is a rapidly advancing area of scientific investigation, as more and more bioactive lipids and their corresponding cell surface receptors are being identified and their actions characterized. Lysophosphatidic acid (1 -acyl-2-hydroxy-sn-glycero-3-phosphate, LPA), the simplest of all glycerophospholipids, represents an important addition to the growing list of lipid messengers. While LPA has long been known as a precursor of phospholipid biosynthesis in both eukaryotic and prokaryotic cells, only recently has it emerged as an intercellular signaling molecule that is rapidly produced and released by activated cells, for example platelets, to influence target cells by acting on a specific cell-surface receptor (Moolenaar (1994) Trends Cell Biol. 4:213-219a0. Other cellular activities elicited by LPA include cell proliferation, chemotaxis, platelet aggregation, and smooth muscle contraction.

Within the nervous system, LPA has been shown to induce growth cone collapse and cell death in postmitotic neurons (Ye et al., 2002, Neuroreport 13:2169-2175), whereas conversely in oligodendrocytes, Schwann cells and astrocytes, LPA promotes cell proliferation and survival (Steiner et al., 2002, Biochim Biophys Acta 1582:154-160). In developing brain, the LPA1 receptor is abundantly expressed in the ventricular zone of the cerebral cortex. LPA1 receptor expression is known to be restricted to the period of neuroblast differentiation into mature neurons (Hecht and Chun, 1996, J Cell Biol 135:1071-1083). Similarly, Schwann cells are known to express the LPA1 receptor only within a limited period of differentiation from the precursor to myelinated cells (Stankoff et al., 2002, Mol Cell Neurosci 20:415-428).

Cellular receptors responsive to LPA compounds have been described, including those designated as LPA1, LPA2, and LPA3 receptor subtypes, formerly known as EDG2, EDG4 and EDG7, respectively (Hecht and Chun, 1996, J Cell Biol 135:1071-1083; An et al., 1998 J Biol Chem 273:7906-7910; Bandoh et al., J Biol Chem 274: 27776-27785). In the practice of the invention, the effect of LPA can be mediated through one or more of these receptors, or other receptors which produce the same biological responses in neural stem/progenitor cells following binding of. LPA.

Any suitable chemical formula for an LPA compound may be used in a tissue culture system according to the invention. Studies of biological activities of LPA compounds have revealed that biological activity typically requires a lengthy acyl carbon chain, for example one comprised of 16 to 18 carbons (Jalink K et al., Biochem. J. 1995, 307:609-16.) Thus a preferred form of an LPA compound of use in the invention can be for example in the range of LPA 20:5 to 8:0, more preferably in the range of LPA 18:0, 16:0, and 14:0 (myristoyl), and most preferably LPA 18:1 (oleoyl) or 16:0 (palmitoyl). An exemplary LPA compound having the 18:1 form is LPA Cat. #857130, from Avanti Polar Lipids (Alabaster, Ala.).

Methods for modifying an LPA compound, for example by ether linkage, by modification of the glycerol backbone, or replacement of the phosphate group, for example by a hydrogen or methyl-phosphonate moiety, are known in the art. Permissible modifications can be determined empirically, for example by testing the biological activity of a candidate LPA compound in a cellular assay in which the test compound is used to elicit a known biological response (such as activation of a signaling cascade, or stimulation of growth and/or differentiation of a neurosphere) in response to activation of an LPA receptor.

In addition to LPA, other lysophospholipid growth factors may be used, such as phospholipid sphingosinel-phosphate (SlP), sphingosyl phosphorylcholine (SPC), psychosine, monoacylglycerol, and anandamide.

Although a number of commercially available basal media (e.g., IMDM, RMPI1640, DMEM, and DMEM/F12) might be used in the invention, those previously shown to support stem cell cultures are preferred. In particular, a methyl cellulose (MC)-based DMEM/F12 media formulation containing insulin is preferred. MC is included in a preferred media formulation as it provides a semi-solid medium. For example, MC-based Dulbecco's Modification of Eagle's Medium/Ham's F-12 (D-MEM, Cat. #10567-014 and F-12 nutrient mixture (Ham), Cat. #31765-035, Gibco BRL, Carlsbad, Calif.) is useful in the invention. Alternatively, an MC-based DMEM/F12 supplement-containing media formulation such as Neurocult media (Cat. #03237, Stem Cell Technologies, Vancouver, BC) is useful in the invention. Such media formulations may be prepared in the laboratory using techniques commonly known in the art, or purchased in a ready-to-use form. Antimicrobials such as antibiotics and antibiotic/antimycotics (for example, Cat. #15240-096, Gibco BRL, Carlsbad, Calif.), antifungal compounds, and/or antiviral compounds can be added to the base medium to prevent contamination of the cultures. Insulin (for example, Cat. #I5523, Sigma, St. Louis, Mo.) can be added to the medium to support the growth of stem cells. To induce cellular differentiation, serum can be added to the basal medium containing LPA. A preferred serum is fetal bovine serum (FBS) (for example, Cat. #16000-044, Gibco BRL, Carlsbad, Calif.).

Preferred concentrations of each of the foregoing are in the following ranges: 1 μM to 50 μM (for example, 10 μM) LPA, 0.4% to 1.6% (for example, 0.8%) MC, 2.5 μg/ml to 10 μg/ml (for example, 5 μg/ml) insulin, 5 ng/ml to 20 ng/ml (for example, 10 ng/ml) EGF, 5 ng/ml to 20 ng/ml (for example, 10 ng/ml) FGF2, 0.25% to 5.0% (for example, 0.5%) FBS, and 1-10 μg/ml (for example, 5 μg/ml) insulin. A preferred concentration of antibiotic/antimycotic is that used according to standard tissue culture procedures.

### Culturing a Neurosphere from Isolated Brain Cells

In a method of culturing a neurosphere from isolated brain cells, an isolated brain cell is provided and cultured in medium containing an LPA compound under conditions that allow for growth of a neurosphere. Brain cells can be isolated and cultured using any suitable technique. For example, techniques described in Kukekov et al., Glia 21:399-406, 1997 and Kukekov et al., Exp. Neurol. 156:333-344, 1999 are useful. Adaptations of these methods involving the following modifications are particularly useful. A mammalian (e.g, mouse, human) brain sample (e.g., approximately 10 mm³) is minced, transferred to a beaker containing a solution of protease (for example, 0.25% trypsin) in 0.1 mM EDTA (for example a mixture of 4:1), and slowly stirred on a magnetic plate at room temperature for a suitable period of time (for example, 15 minutes). After trituration through a plastic 5 ml pipette and a fire-polished Pasteur pipette, the dissociate is washed (for example, 5-8 times in 5 ml of medium) to eliminate cellular debris. The resultant suspension is filtered through sterile gauze and confirmed contain only single cells and counted with a hemocytometer. Cells are then resuspended in 0.8% MC-based insulin-containing medium (for example, NeuroCult, Cat. #03237, StemCell Technologies, Vancouver, BC) and plated at a clonal density (for example, 105 cells per well) in multi-well (for example, 6-well) plates coated with a non-adhesive substrate such as poly-HEME (poly 2-hydroxyethyl methacrylate, Sigma, St. Louis, Mo.) according to manufacturer's instructions. Fresh aliquots of growth factor(s) (for example, LPA (10 μM) or LPA in combination with other growth factors, for example EGF+FGF2+LPA are added every 3 days during the 21 days of neurosphere generation. Cells are maintained in a 37° C. incubator with 95% air, 5% CO₂ and 100% humidity. Neurospheres typically become visible under an inverted phase microscope at approximately one week post-plating.

### Differentiation of Neurospheres into Neurons, Astrocytes and Oligodendrocytes

The method of culturing a neurosphere from isolated brain cells further allows for proliferation and differentiation of the neurosphere into neurons, astrocytes and oligodendrocytes. To induce a neurosphere to differentiate into neurons, astrocytes and oligodendrocytes, the neurosphere is cultured under conditions that promote cellular differentiation. In a preferred method of inducing neurospheres to differentiate, neurospheres are cultured in a standard medium containing serum. In one example of this method, neurospheres are placed on a coverslip coated with laminin and poly-L-ornithine in the presence of serum. Culture conditions for differentiating neural stem cells into astrocytes, neurons and oligodendrocytes are discussed, for example in Weiss et al., U.S. Pat. No. 5,851,832; Weiss et al., U.S. Pat. No. 6,497,872; and Capela and Temple, Neuron 35:865-875; Oishi et al., J. Physiol. 540:139-152, 2002; Kukekov et al., Exp. Neurol. 156:333-344, 1999; and Laywell et al., Exp. Neurol. 156:430-433, 1999.

To confirm the differentiation of cells into neurons, astrocytes and oligodendrocytes, the cells can be subjected to reverse-transcriptase polymerase chain reaction (RT-PCR) to determine the presence of-cell-specific markers (for example, GFAP for astrocytes, beta III tubulin for neurons, and O4 for oligodendrocytes). Alternatively, antibodies specific for various neuronal or glial proteins may be employed to identify the phenotypic properties of the differentiated cells using methods such as immunocytochemistry. Neurons may be identified using antibodies to neuron-specific enolase, neurofilament, tau, beta-tubulin, or other known neuronal markers. Astrocytes may be identified using antibodies to GFAP or other known astrocytic markers. Oligodendrocytes may be identified using antibodies to galactocerebroside, O4, myelin basic protein or other known oligodendrocytic markers. Glial cells in general may be identified by staining with antibodies, such as the M2 antibody, or other known glial markers. It is also possible to identify cell phenotypes by identifying compounds characteristically produced by those phenotypes. For example, neurons may be identified by their production of neurotransmitters, such as acetylcholine, dopamine, epinephrine, norepinephrine and the like.

### Use of Neural Stem/Progenitor Cells

The cultured neural stem cell/progenitor cells of the invention are useful in a variety of ways. The cells can be used to reconstitute a host whose cells have been lost through disease or injury. Genetic diseases associated with cells may be treated by genetic modification of autologous or allogeneic stem cells to correct a genetic defect or to protect against disease. Alternatively, normal allogeneic progenitor cells may be transplanted. Diseases other than those associated with cells may also be treated, where the disease is related to the lack of a particular secreted product such as hormone, enzyme, growth factor, or the like.

CNS disorders encompass numerous afflictions such as neurodegenerative diseases (e.g. Alzheimer's and Parkinson's), acute brain injury (for example, stroke, head injury, cerebral palsy) and a large number of CNS dysfunctions (for example, depression, epilepsy, and schizophrenia). In recent years neurodegenerative disease has become an important concern due to the expanding elderly population which is at greatest risk for these disorders. These diseases, which include Alzheimer's Disease, multiple sclerosis (MS), Huntington's disease, amyotrophic lateral sclerosis (ALS), and Parkinson's disease, have been linked to the degeneration of neural cells in particular locations of the CNS, leading to the inability of these cells or the brain region to carry out their intended function. By providing for maturation, proliferation and differentiation into one or more selected lineages through specific different growth factors the stem/progenitor cells of the invention may be used as a source of committed cells. The cells and methods of the invention are intended for use in a mammalian host, recipient, patient, subject or individual, preferably a primate, most preferably a human.

### EXAMPLES

The present invention is further illustrated by the following specific examples. The examples are provided for illustration only and are not to be construed as limiting the scope or content of the invention in any way.

### Example 1

### Methods for Cultivation of Neurospheres in Vitro

Preparation of single-cell suspensions and cloning in MC. Dissociates of postnatal (5-7 days old) C57/BL mouse forebrain (subependymal zone and hippocampus) were used to generate suspended clones under anchorage and serum withdrawal in semi-solid methyl cellulose (MC). The procedures were performed as previously described Kukekov et al., Glia 21:399-407, 1997; and Kukekov et al., Exp Neurol, 156:333-344, 1999, with some additional modifications. Brain samples (approximately 10 mm³) were minced, transferred to a beaker containing a solution of 0.25% trypsin in 0.1 mM EDTA (mixture 4:1), and slowly stirred on a magnetic plate at roomm temperature for 15 minutes. After trituration through a plastic 5 ml pipette and a fire-polished Pasteur pipette, the dissociate was washed (5-8 times in 5 ml of medium) to eliminate cellular debris.

The resultant suspension was filtered through sterile gauze, and verified to contain only single cells which were counted with a hemocytometer. Finally, cells were resuspended in 0.8% MC-based medium (NeuroCult medium, Cat. #03237, StemCell Technologies, Vancouver, BC) and plated at a clonal density (i.e., about 10⁵ cells per well) in 6-well plates coated with a non-adhesive substrate, i.e., poly 2-hydroxyethyl methacrylate (poly-HEME, Sigma) according to the manufacturer's instructions. Cells were maintained in a 37° C. incubator with 95% air, 5% CO₂, and 100% humidity. Clones became visible under an inverted phase microscope approximately one week after plating.

### Example 2

### Quantitative Measurements of Neurospheres

Morphometric analysis of neurospheres. The number and diameters of neurospheres was assessed using an inverted phase microscope equipped with a size covering net. The number of neurospheres in each experimental group was normalized to the protein content. Aliquots of suspensions were removed for protein measurement using Pierce BCA protein Kit (Pierce, Rockford, Ill.) according to the manufacturer's instructions. Measurements were taken following one, two and three weeks of growth in vitro.

### Example 3

### Methods for Determining Neurosphere Viability and Cell Proliferation

Cell viability is determined by standard techniques, for example by measuring conversion of a tetrazolium salt (MTS) into an insoluble dye according to the manufacturer's instructions (MTS Assay, Promega, Madison, Wis.). MTS stock solution (5 mg/ml) is added to wells containing clones on cover slips and incubations are continued for 2 to 3 hours to cultured cells (in 1/10 of the original culture volume), then 100 μl aliquots of medium are removed. The converted dye is solubilized with acidic isopropanol. Absorbance is measured at a wavelength of 570 nm with a background subtraction at 630 nm.

Proliferative activity of cells in neurospheres is assessed by measuring DNA synthesis using [³H]-thymidine incorporation, for example at three time points during neurosphere growth such as at one week, two weeks and three weeks. Neurospheres are removed from MC, and then transferred in a drop of DMEM/F12 medium without serum or growth factors to glass cover slips sequentially coated with poly-L-ornithine (1 mg/cm², #P-3655 Sigma, St. Louis, Mo.) and laminin (0.5 mg/cm², #L-2020 Sigma, St. Louis, Mo.) (P/L), and placed in 12-well plates (Corning, Corning, N.Y.) at a density of 10-20 clones per cover slip. After 4-6 hrs, one ml of DMEMIF12 medium with 0.5% FBS is added to each P/L cover slip with the attached MC-PL clones to allow the cells to express differentiation features. One day, one week and two weeks later, cover slips with the individual MC-PL clones are processed for [³H]-thymidine incorporation as follows [³H]thymidine(1 μCi/well) is added to several randomly selected wells and cells are cultured for an additional 24 hours under the same conditions. An aliquot of cells is collected by centrifugation, gently rinsed with PBS, ice-cold 10% trichloroacetic acid (TCA) and then thoroughly washed with ice-cold 90% ethanol. Cells are lysed with 1% Triton X-100 and radioactivity is counted in a liquid scintillation counter.

### Example 4

### Cultivation of Neurospheres Using Growth Factors

Using the above-described methods, dissociates of postnatal(5-7 days old) C57/BL mouse forebrain, (subependymal zone and hippocampus) were used to generate suspended clones under anchorage and serum withdrawal in semi-solid MC (MC-clones) Equal amounts of dissociates were placed in serum-free DMEM/F12, 1:1 medium supplemented with 0.8% MC and insulin (NeuroCult medium, Cat. #03237, StemCell Technologies, Vancouver, BC) in the presence of (i) 10 ng/ml EGF plus 10 ng/ml FGF2, (ii) 10 μM LPA alone and (iii) EGF+FGF2 in combination with LPA. Growth factors, i.e., EGF, FGF2 were obtained from Peprotech, Inc., and LPA was obtained from LPA-Avanti Polar Lipids, Inc. Freshly prepared aliquots of EGF and FGF2 were added every 3 days during a 21 day interval of clone (neurosphere) generation. The numbers and diameters of neurospheres were measured at the end of the third week in vitro.

### Example 5

### Immunocytochemical Staining of Neurospheres

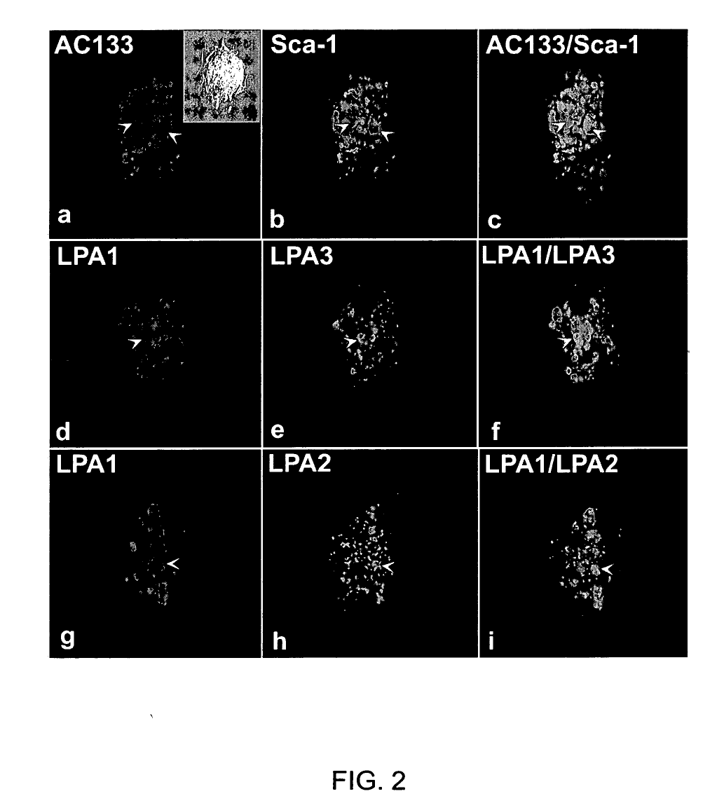

Neurospheres were grown in suspension in the presence of LPA for 18 days, then transferred onto cover slips coated with laminin and poly-L-ornithine. The neurospheres attached within 6 hours and were allowed to spread and differentiate for an additional 48 hours. Cover slips were then fixed and processed for immunostaining. Antibodies used in these studies included antibodies against AC133 antigen (Miltenyi Biotech) and Sca-1 antigen (BD Pharmingen), against LPA receptors LPA1, LPA2 and LPA3 Exalpha Biologicals Inc., Antibody Solutions), βIII-tubulin (ovance, Inc.), and nestin (Chemicon, Inc.), CNPase (Sigma). To study the effect of LPA on differentiation of neurospheres, in some cases double immunostaining of LPA receptors and neuronal markers was performed. Results were analyzed using confocal microscopy. Visualization of the proteins was performed by two-color fluorescent dye labeling (i.e., red-Texas Red, green-Alexa Green) and subsequent analysis in laser confocal microscope appropriately equipped with filters of different wavelengths. The images were taken at the same gain setting for each filter within one experimental set. As an example, referring to FIG. 2A, B, the presence of AC133 antigen was detected by red fluorescence and that of Sca-1 by green fluorescence using separate wavelength visualization. AC133/Sca-1 co-localization was detected as yellow fluorescence using dual wavelength visualization (shown in right column of FIG. 2).

### Example 6

### LSA Induces Formation and Supports Growth of Neurospheres

This example presents results of studies of neurospheres isolated from mouse postnatal brain and cultivated in the presence of LPA, peptide growth factors EGF and bFGF, and a combination of LPA, EGF and bFGF, revealing that LPA alone effectively supports growth of neurospheres.

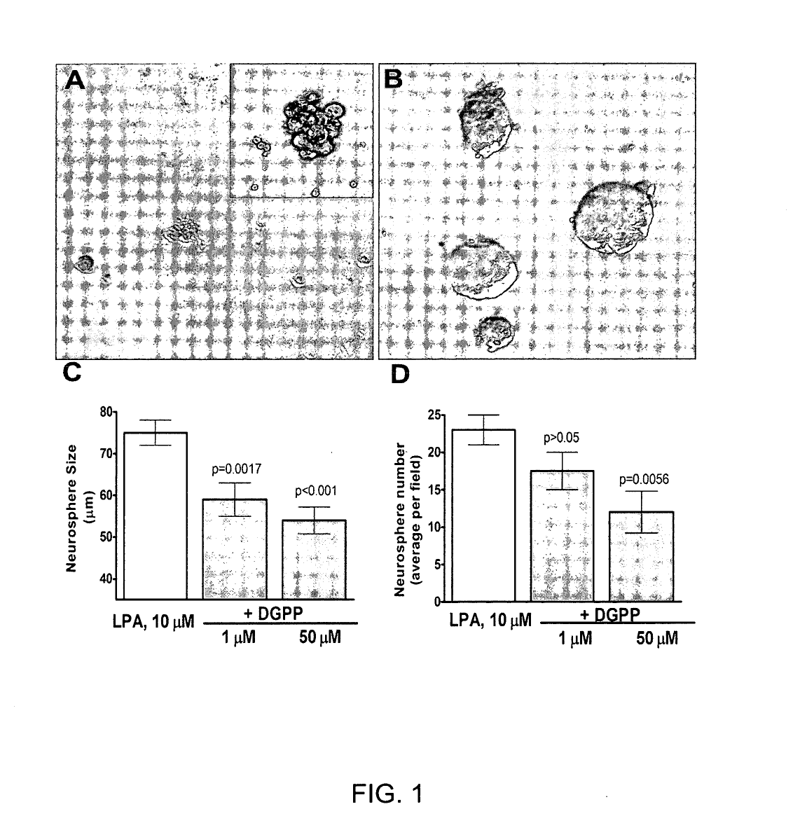

Using methods described above, cells were recovered after complete dissociation of mouse forebrain to single cells and plated at a density of ˜10⁴ cells/well in serum free semi-solid methylcellulose medium containing growth factors, ie., 10 μM oleoyl (C18:1) LPA, or 10 ng/ml each of EGF and FGF2. Addition of methylcellulose to the medium prevented lateral diffusion of the cells and formation of cell aggregates within one hour after plating as assessed by optical microscopy ((Kukekov et al., Glia 21:399-407, 1997; Kukekov et al., Exp Neurol, 156:333-344, 1999). As seen in FIG. 1A, 72 to 96 hours after initiation of the cultures, cells maintained in the presence of LPA or EGF+FGF2 formed small aggregates consisting of a few cells. These bodies continued to grow and formed distinguishable neurospheres during the first week in culture. In cultures treated without growth factors, very few cell aggregates formed, and mostly tissue debris was seen in the cultures. Neurosphere growth persisted during the second week in semi-solid medium continuously supplemented with LPA or EGF+FGF2 (FIG. 1B).

Referring to Table 1, the number and diameter of neurospheres was assessed in cultures propagated in the presence of the various growth factors. The results demonstrated that LPA generated aprroximately the same number of neurospheres per field as did EGF+FGF2 (i.e., 220 vs. 199). The diameter of neurospheres produced by LPA was significantly smaller than observed in the presence of EGF+FGF2 (i.e., 79.54 vs. 115.5 μm).

TABLE 1

| Growth Factor | EGF + FGF | LPA |
|---|---|---|
| Mean | 115.5030151 | 79.54227273 |
| Standard Error | 3.989879328 | 2.758648414 |
| Median | 103.8 | 72.25 |
| Mode | 95.5 | 55.4 |
| Standard Deviation | 56.28417427 | 40.91736839 |
| Sample Variance | 3167.908274 | 1674.231036 |
| Kurtosis | −0.314252562 | 0.484977379 |
| Skewness | 0.687998228 | 0.874354091 |
| Range | 260.8 | 189.9 |
| Minimum | 12.9 | 14.1 |
| Maximum | 273.7 | 204 |
| Sum | 22985.1 | 17499.3 |
| Count | 199 | 220 |
| Largest (1) | 273.7 | 204 |
| Smallest (1) | 12.9 | 14.1 |
| Confidence Level | 7.868107607 | 5.436901065 |
| (95.0%) | | |

The growth of neurospheres maintained by EGF+FGF2 was maximal after 4 to 5 weeks in vitro and gradually declined thereafter, with concomitant neurosphere degradation, cell death and formation of debris. In marked contrast, LPA-dependent neurosphere development was sustained for up to 3 months.

### Example 7

### Role of LPA Receptors in Formation and Differentiation of Neurospheres

The biological effect of LPA is mediated through specific LPA receptors such as LPA1, LPA2 and LPA3 (Hecht and Chun, 1996, J Cell Biol 135:1071-1083; An et al., 1998 J Biol Chem 273:7906-7910; Bandoh et al., J Biol Chem 274:27776-27785) Short chain diacyl-glycerol pyrophosphate (C8-DGPP) at low concentration is known to preferentially inhibit the LPA3 receptor, while at high concentration it blocks LPA activation of both LPA1 and LPA3 receptor subtypes (Sardar et al., 2002 Biochim Biophys Acta 1582:309-317). To examine the potential involvement of LPA receptors in the LPA-induced formation of neurospheres, the effect of DGPP at low concentration (i.e., 1 μM) and high concentration (i.e., 50 μM) was compared.

Referring to FIG. 1C, the results showed that DGPP at a concentration of either 1 μM or 50 μM significantly reduced the size of neurospheres generated by LPA By contrast, the numbers of neurospheres formed was decreased considerably by 50 μM DGPP (p=0.0056), but not by DGPP at 1 μM concentration (FIG. 1D). The data thus indicated that the initial formation of neurospheres by LPA is predominantly mediated by the LPA1 receptor, whereas the subsequent cell propagation and neurosphere growth are dependent on both LPA1 and LPA3 receptor activation.

### Example 8

### LPA-Generated Neurospheres Co-Express AC133 and Sca-1 Antigens, and LPA Receptors

Neurospheres were grown using methods described above in the presence of LPA in semi-solid medium for two weeks. Aliquots of neurospheres were then removed and placed onto glass cover slips coated with laminin/poly-L-ornithine. Fresh medium without methyl cellulose and FBS (10% final) were added to initiate neurosphere adherence to the cover slip surface. The neurospheres became completely attached within 4 to 6 hours after transfer. The appearance of a typical neurosphere observed at this time point is shown in the inset in FIG. 2A.

Cover slips were then fixed, stained with antibodies against AC133, Sca-1, LPA1, LPA2 and LPA3 receptors and analyzed using immunofluorescent techniques, as described above. Referring to FIG. 2A-C, the neurospheres expressed both AC133 and Sca-1 antigens, which are markers of the most primitive stem/progenitor cells. AC133 and Sca-1 co-localization was highly reproducible and consistent in neurospheres of different size. Nearly 100% co-localization of AC133 and Sca-1 was observed throughout the neurospheres, with the most extensive expression occurring in the core of the neurosphere (indicated by arrowheads in FIGS. 2A-C). Distribution of these markers was strictly co-localized throughout the neurosphere with the presence of LPA.

Referring to FIG. 2D-I, these cells were seen to exhibit LPA1, LPA2 and LPA3, i.e., all three subtypes of receptor for LPA. The LPA1 , LPA2 and LPA3 receptors were cross co-localized, with substantial accumulation in the neurosphere core (FIG. 2F and 2I arrowheads). As can be seen in the figure, LPA1, LPA3 and, particularly LPA2 receptor distribution was not uniform within the neurosphere. In addition, the magnitude of LPA receptor accumulation varied slightly in different neurospheres of the same preparation.

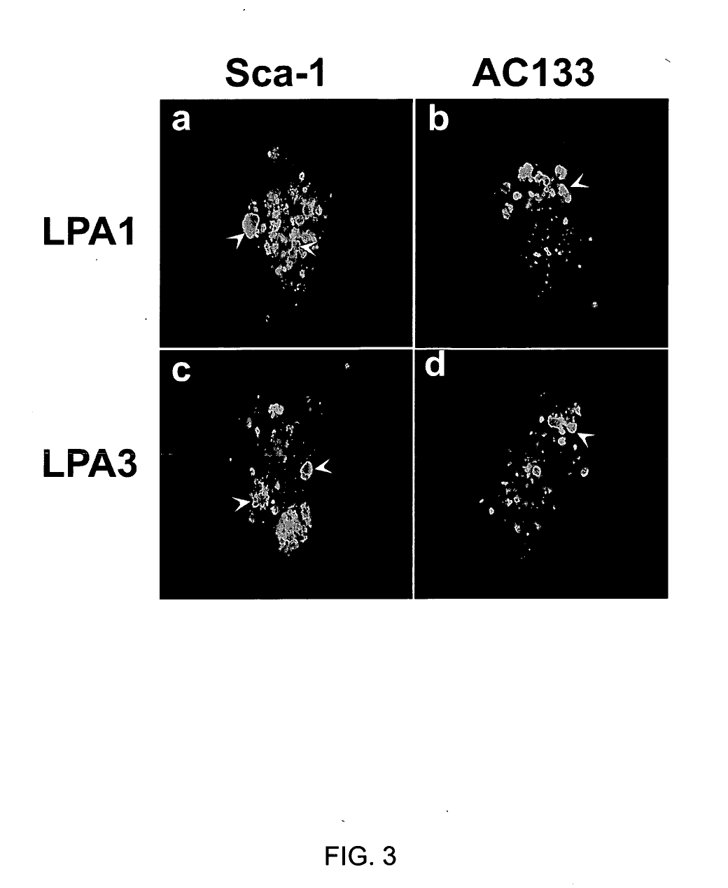

Referring now to FIG. 3, LPA receptors in the grown neurosphere were expressed in the cells harboring AC133 and Sca-1 antigen as judged by a focal co-localization of LPA1 and LPA3 with both AC133 and Sca-1 within the neurosphere. A majority of AC133 and Sca-1, positive cells exhibited a comparable level of LPA1 and LPA3 receptor expression (FIG. 3, arrowheads), which was almost uniformly distributed in co-localization zones within the neurosphere. However, the prevalence of LPA1 -positive expression could be found in Sca-1 and AC133 co-localization areas, respectively (FIG. 3A, B) as well as LPA3/AC133-co-expressing cells.

### Example 9

### Co-Expression of Lineage-Specific Markers and LPA Receptors in Differentiating Stem/Progenitor Cells of Neurospheres

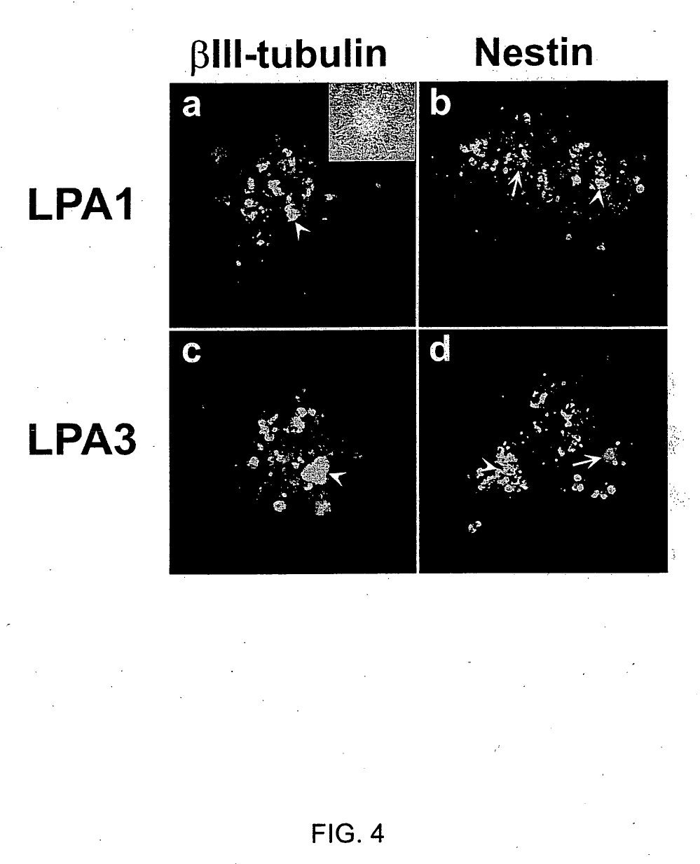

Neurospheres were grown in the presence of LPA as described for two weeks in semi-solid medium. Aliquots were then transferred onto cover slips coated with laminin/poly-L-ornithine, attached in the presence of serum, and maintained in serum-free medium supplemented with LPA for an additional two weeks. The cells migrated from the core of the neurosphere and differentiated during this time. Referring to the inset in FIG. 4A, a typical neurosphere is shown after growth for two weeks following attachment in the presence of LPA. Although the cells consisting the core of the initial neurosphere spread significantly, the spheres still retained a dense cellular core containing several cellular layers.

Neurospheres grown attached for two weeks expressed βIII-tubulin, a marker of immature neurons, predominantly in the residual core of the neurosphere (FIG. 4A and 4C, arrowheads), which was co-localized with LPA1 and LPA3 receptors. A significant level of expression of nestin was also found in attached neurospheres, which correlated with LPA1 and LPA accumulation by double immunocytochemical staining (FIG. 4B and D, arrowheads). Separate localization of nestin and LPA1 receptor was also observed in double-stained neurospheres. In addition, expression of LPA3 receptors occurred in the cells, which did not exhibit significant amount of nestin (FIG. 4D, arrow).

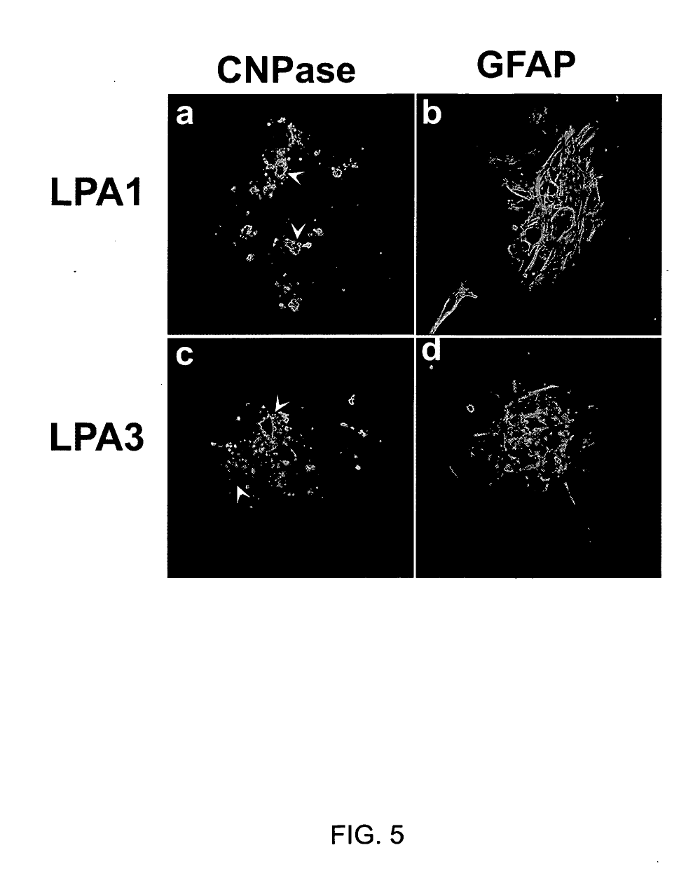

The cells induced to differentiate by attachment of neurospheres to cover slips and maintenance in the presence of LPA displayed phenotypic characteristics of glial lineages. Both CNPase and GFAP, markers of oligodendrocytes and astrocytes respectively, were found to express in developing neurospheres (FIG. 5). Expression of LPA receptors occurred in CNPase-positive cells as indicated by double immunostaining (FIGS. 5A and 5C, arrowheads). There was a substantial prevalence of CNPase accumulation over LPA1 and LPA3, while separate expression of LPA1 was observed in several cells of neurospheres. In contrast, the cells which migrated outside the neurosphere core and which expressed the astrocyte lineage marker GFAP did not accumulate any significant amount of LPA1 or LPA3 receptors.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A tissue culture system comprising:
(a) at least one isolated neural cell expressing at least one LPA receptor;
(b) a lysophosphatidic acid (LPA) compound; and
(c) a basal culture medium.

2. The tissue culture system of claim 1, wherein the form of said LPA compound is selected from the group consisting of LPA 20:5, 18:1 (oleoyl), 16:0 (palmitoyl), and 14:0 (myristoyl).

3. The tissue culture system of claim 2, wherein the form of said LPA compound is 18:1 (oleoyl) or 16:0 (palmitoyl).

4. The tissue culture system of claim 1, wherein said isolated neural cell is a stem/progenitor cell.

5. The tissue culture system of claim 4, wherein said neural stem/progenitor cell is situated within a neurosphere.

6. The tissue culture system of claim 4, wherein said neural stem/progenitor cell is derived from a mammal.

7. The tissue culture system of claim 6, wherein-said-mammal is a mouse.

8. The tissue culture system of claim 6, wherein said mammal is a human.

9. The tissue culture system of claim 1, wherein said LPA receptor expressed by said neural cell is selected from the group consisting of an LPA1, LPA2, and LPA3 receptor.

10. The tissue culture system of claim 1, wherein said stem/progenitor cell expresses at least one of a Sca-1 and an AC133 antigen, and at least one of an LPA1, LPA2 and LPA3 receptor.

11. The tissue culture system of claim 10, wherein said stem/progenitor cell further expresses at least one marker of neuronal differentiation selected from the group consisting of βIII tubulin, and nestin.

12. A method of culturing at least one neurosphere from isolated brain cells, the method comprising the steps of:
(a) providing at least one isolated brain cell; and
(b) culturing said at least one brain cell in a medium containing a lysophosphatidic acid (LPA) compound under conditions that allow for growth and differentiation of a neurosphere from said isolated brain cell.

13. The method of claim 12, wherein the step (b) of culturing the at least one brain cell under conditions that allow for growth of a neurosphere further allows for proliferation and differentiation of the cells within said neurosphere into at least one cell type selected from the group consisting of a neuron, an astrocyte and an oligodendrocyte.

14. The method of claim 13, wherein said at least one cell type is a neuron, wherein at least one lineage-specific marker is expressed by said cell, said marker selected from the group consisting of βIII tubulin and nestin.

15. An isolated neural cell cultivated in a basal culture medium comprising a lysophosphatidic acid (LSA) compound.

16. The isolated neural cell of claim 15, wherein said cell is a stem/progenitor cell.

17. The isolated neural cell of claim 15, wherein the form of said LPA compound is selected from the group consisting of LPA 20:5, 18:1 (oleoyl), 16:0 (Palmitoyl), and 14:0 (myristoyl).

18. The isolated neural cell of claim 17, wherein the form of said LPA compound is LPA 18:1 (oleoyl) or LPA 16:0 (palmitoyl).
